(19) United States
(12) Patent Application Publication
(10) Pub. No.: US 2024/0041343 A1
(43) Pub. Date: Feb. 8, 2024

(54) SYSTEM AND METHOD FOR ESOPHAGEAL HYDROMETRY

(71) Applicant: Northwestern University, Evanston, IL (US)

(72) Inventors: John Erik Pandolfino, Evanston, IL (US); Dustin Allan Carlson, Evanston, IL (US); Wenjun Kou, Evanston, IL (US)

(21) Appl. No.: 18/546,592

(22) PCT Filed: Feb. 16, 2022
(86) PCT No.: PCT/US2022/016661

§ 371 Date: Aug. 15, 2023

Related U.S. Application Data
(60) Provisional application No. 63/150,560, filed on Feb. 17, 2021.

(51) Int. Cl. A61B 5/03; A61B 5/00; A61M 25/10; A61B 5/11

(52) U.S. Cl. CPC A61B 5/037; A61B 5/7267; A61M 25/1011; A61B 5/1107; A61B 5/687; A61B 5/6853; A61B 2562/0247

(57) ABSTRACT

A catheter-based baro-hydrometer device measures or otherwise monitors esophageal function. The device includes a segmented balloon design, including a first and second balloon whose fill volumes can be independently controlled. In general, the device leverages secondary peristalsis related to balloon distention in order to study esophageal function. Pressure sensors coupled to the balloons record pressure data, which can be classified and processed to measure or otherwise monitor esophageal function.

### CROSS-REFERENCE TO RELATED APPLICATIONS

This application claims the benefit of U.S. Provisional Patent Application Ser. No. 63/150,560, filed on Feb. 17, 2021, and entitled “SYSTEM AND METHOD FOR ESOPHAGEAL HYDROMETRY,” which is herein incorporated by reference in its entirety.

### STATEMENT REGARDING FEDERALLY SPONSORED RESEARCH

This invention was made with government support under DK117824 awarded by the National Institutes of Health. The government has certain rights in the invention.

### BACKGROUND

Studying how the esophagus reacts to distention is crucial to understanding esophageal diseases, as the primary goal of the esophagus is to move fluid and food into the stomach during swallowing and after reflux events. The tools focused on this approach have limitations, such as requiring the patient to be awake (manometry), or not being able to control the response while the patient is sedated during endoscopy.

### SUMMARY OF THE DISCLOSURE

The present disclosure addresses the aforementioned drawbacks by providing a baro-hydrometer device that includes a catheter extending from a first end to a second end along a longitudinal axis, a first balloon coupled to the catheter, and a second balloon coupled to the catheter. The first balloon is arranged at the first end of the catheter, and the second balloon arranged at the second end of the catheter, where the second balloon is smaller than the first balloon. A first infusion port fluidically couples the first balloon to the catheter, and a second infusion port fluidically couples the second balloon to the catheter. A plurality of pressure sensors are arranged on the first balloon and the second balloon.

It is another aspect of the present disclosure to provide a method for measuring esophageal function in a subject. The method includes positioning a catheter within an esophagus of the subject, where the catheter includes a first balloon having a pressure sensor coupled thereto and a second balloon having a pressure sensor coupled thereto. Pressure data are measured with the pressure sensors coupled to the first and second balloons while independently controlling a fill volume of the first balloon and a fill volume of the second balloon. Classified pressure data are generated by inputting the pressure data to a classifier implemented with a computer system. Esophageal function data are then generated from the classified pressure data using the computer system.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is an example catheter-based baro-hydrometer device for measuring esophageal function.

FIGS. 2A-2D show example pressure recording measured using a baro-hydrometer device.

FIG. 3 is a flowchart setting forth the steps of an example method for generating esophageal function data using a baro-hydrometer device.

FIGS. 4A-4K show example of pressure profiles indicating different pressure patterns that can be used to construct and/or train a classifier model, such as a machine learning algorithm or model.

FIG. 5 is a block diagram of an example system for generating esophageal function data from pressure data measured using a baro-hydrometer device.

FIG. 6 is a block diagram of example components that can implement the system of FIG. 5.

### DETAILED DESCRIPTION

Described here is a device for measuring or otherwise monitoring esophageal function during manometry, and methods for its use. Advantageously, the device can be used while a patient is sedated. In general, the device leverages secondary peristalsis related to balloon distention in order to study esophageal function while patients are sedated during manometry. In some embodiments, the device may be referred to as a baro-hydrometer device.

The baro-hydrometer device generally includes a catheter that is configured to elicit a patterned response in the esophagus, including relaxation and secondary peristalsis. Pressure changes within a fluid filled bag are then measured and converted to tracings in order to determine a pressure inversion point and propagation of peristalsis in the esophagus. The overall pressure generated by the bag along with periods of equalization of pressure can be used to characterize the function of the esophagus.

The baro-hydrometer device described in the present disclosure uses a model of short segmental balloon stimulation while the patient is sedated in order to elicit two responses in the esophagus: relaxation of the esophagus and secondary peristalsis. This model allows for a more cost-effective approach as it does not require the use of more expensive impedance planimetry technology. The measured pressure response data can be classified using a suitable classifier or trained machine learning algorithm in order to predict esophageal function. For instance, unique pressure signals that can be measured with the baro-hydrometer device correspond to, or otherwise can be correlated with, specific patterns of motility. In this way, the systems and methods described in the present disclosure provide a simple way to perform a motility assessment during an endoscopic procedure (e.g., index endoscopy).

In general, the baro-hydrometer device includes an additional bag (e.g., balloon) with a fluid infusion (e.g., an air infusion) that is used to stimulate motility while the hydrometer bag is empty, thereby allowing for motility testing during sedated endoscopy without asking the patient to swallow. This approach allows for a controlled assessment with the additional balloon eliciting contractions with short fill and empty protocols.

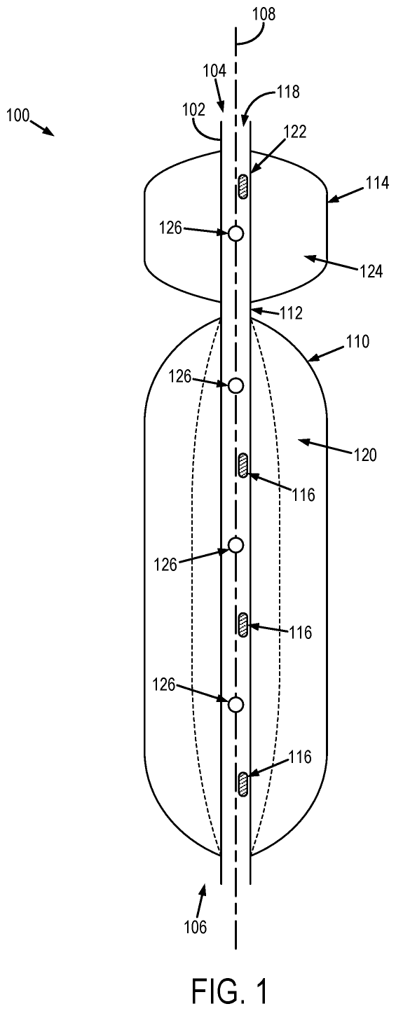

Referring now to FIG. 1, an example baro-hydrometer device 100 in accordance with some embodiments described in the present disclosure is shown. In this configuration, the baro-hydrometer device 100 generally includes a catheter 102 extending from a proximal end 104 to a distal end 106 along a central longitudinal axis 108. In some configurations, the catheter 102 can have a 5 mm diameter. Alternatively, the catheter 102 can have other diameters suitable for use in esophageal applications, such as in a range of 2 mm to 8 mm. The catheter 102 can be composed of a suitable material, such as a soft medical-grade, clear polyvinyl chloride (“PVC”) tubing, thermoplastic elastomers (“TPEs”), and/or polypropylene (“PP”).

A first balloon 110 is coupled to or otherwise arranged on the external surface 112 of the catheter 102. In a non-limiting example, the first balloon 110 can have a length of 16 cm along the direction of the longitudinal axis 108 of the catheter 102, and can have a maximum diameter of 24 mm in its expanded state and a fill volume of 70 mL. It will be appreciated that the first balloon 110 may also be configured to have a different length, maximum diameter, and/or fill volume depending on the particular application. In some configurations, the first balloon 110 can be composed of a non-latex polyurethane bag.

A second balloon 114 is also coupled to or otherwise arranged on the external surface 112 of the catheter 102. In some configurations, the second balloon 114 is located proximal to the first balloon 110. Alternatively, the second balloon 114 may be located distal to the first balloon 110. Generally, the second balloon 114 is smaller than the first balloon 110. For example, the second balloon 114 can have a maximum diameter of 24 mm, a fill volume of 20 mL, and a length of 3 cm. In general, a shorter (e.g., 3 cm or less) second balloon 114 is contemplated to better convert the volume inflation as focal dilation to trigger secondary peristalsis.

One or more infusion ports 116 provide a fluid connection between the interior lumen 118 of the catheter 102 and the inner volume 120 of the first balloon 110. As a non-limiting example, the infusion port(s) 116 can have a diameter between 1.5 and 2 mm. The fluid can be a general medium appropriate for inflating a balloon that is inserted in the human organ. The properties of the selected fluid medium can impact the registered/measured pressure variations. As one example of how the fluid medium selection will impact the pressure variations, the fluid medium could generate pressure variations associated with the process of filling/emptying of the balloon. This process can be referred to as the transition process. Given the same setup of the catheter configuration shown in FIG. 1, the magnitude of variation will decrease with the increase of time period of transient process the and decrease of friction property of the fluid medium. Hence, to reduce the pressure variation associated with the transition process, air can be a preferable fluid medium due to its low friction. Alternatively, saline could also be used as the fluid medium with a longer transition period.

As another example of how the fluid medium selection will impact the pressure variations, the gravitational force of the fluid medium within the balloon may generate spatial variation of pressure, particularly within the first balloon 110. The magnitude of variation will decrease with the decrease of the fluid medium's density; thus, lighter fluids like air may be preferred.

As still another example of how the fluid medium selection will impact the pressure variations, the dynamics of moving fluid resulting from esophageal contraction and relaxation will also generate pressure variations. The magnitude of these variations is contemplated to be smaller with a lighter fluid medium, such as air.

When fluid medium (e.g., air or saline) is pumped or otherwise provided to the interior lumen 118 of the catheter 102 it will flow into the inner volume 120 of the first balloon 110 via the infusion port(s) 116, thereby expanding the first balloon 110 radially outward from the external surface 112 of the catheter 102. In some configurations, the first balloon 110 can be filled with infinite compliance up to its maximum diameter.

Another infusion port 122 also provides fluid connection between the interior lumen 118 of the catheter and the inner volume 124 of the second balloon 114. As a non-limiting example, the infusion port 122 that supplies the second balloon 114 can have a diameter between 1.5 and 2 mm. When fluid is pumped or otherwise provided to the interior lumen 118 of the catheter 102 it will flow into the inner volume 124 of the second balloon 114 via the infusion port 122, thereby expanding the second balloon 114. In some embodiments, the fluid provided to the second balloon 114 can be air. Air can be advantageously used in the second balloon 114 because it allows for short transition times (i.e., short fill/empty times) on account of its low flow resistance and density. For example, using air as the fluid, transition times on the order of 10 seconds can be achieved. Other low flow resistance and/or low-density fluids could also be used. Alternatively, fluids like saline can also be used, though using a fluid like saline may yield longer transition times and noisier pressure fluctuations.

In some embodiments, the interior lumen 118 of the catheter 102 may include separate channels to provide independent fluid control to the first balloon 110 and the second balloon 114. For example, a first channel can provide fluid flow to the first balloon 110 and a second channel can provide fluid flow to the second balloon 114. In this way, the second balloon 114 can be filled with fluid (e.g., air) to stimulate motility while the first balloon 110 is empty, thereby allowing for motility testing without needing the patient to swallow. Controlled assessment of esophageal function can thus be provided with the second balloon 114 eliciting contractions with short fill and empty protocols. Having two independent balloons advantageously “mirrors” esophageal active functioning like excitation-inhibition, besides the measurement of volume-pressure data.

One or more pressure sensor transducers 126 are coupled to or otherwise arranged on the external surface of the first balloon 110 and the external surface of the second balloon 114. For example, the catheter can be equipped with between 3 and 32 pressure sensor transducers 126, which in some embodiments may be solid state pressure sensors. In other embodiments, a higher number of pressure sensor transducers 126 can also be used to increase the spatial resolution of the pressure measurements. For example upwards of 64 pressure sensor transducers 126, upwards of 128 pressure sensor transducers 126, or so on could also be used. A larger balloon size will allow for a larger number of pressure sensor transducers 126.

When the first balloon 110 and second balloon 114 are deployed within a patient's esophagus and expanded into their respective expanded states, the pressure sensor transducer(s) 126 will make contact with the internal surface of the esophagus. Pressure changes in the patient's esophagus can then be recorded or otherwise measured using the pressure sensor transducer(s) 126. Pressure data measured or otherwise recorded with the pressure sensor transducers 126 can be communicated to a measurement unit, which may in some instances be implemented by a processor, a computer system, or the like.

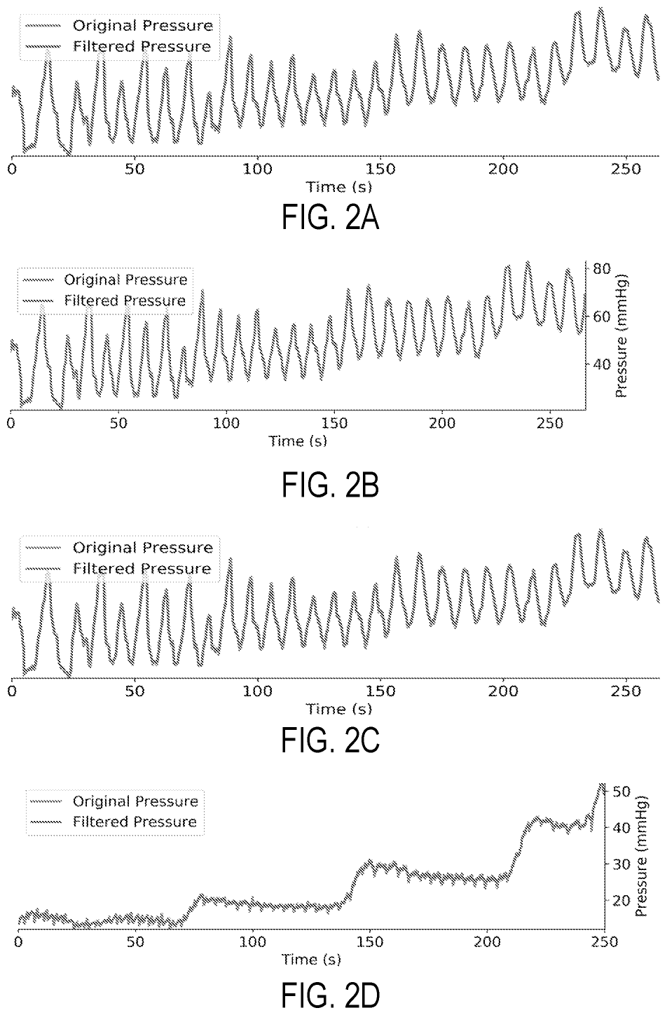

By way of illustration, example pressure data measured with the pressure sensor transducer(s) 126 on the first balloon 110 are shown in FIGS. 2A-2C, and example pressure data measured with the pressure sensor transducer(s) 126 on the second balloon 114 are shown in FIG. 2D.

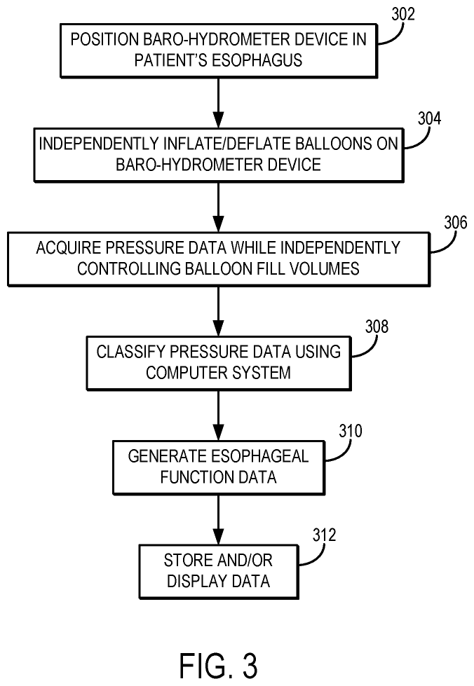

Referring now to FIG. 3, a flowchart is illustrated as setting forth the steps of an example method for generating esophageal function data using a baro-hydrometer device, such as those described in the present disclosure.

The method includes positioning the baro-hydrometer device in an esophagus of a subject, as indicated at step 302. The first and second balloons of the baro-hydrometer device are then inflated and deflated, as appropriate, by providing fluid to each balloon under control of a user, as indicated at step 304. As described above, the inflation and deflation of the first and second balloons can preferably be independently controlled, such that the fill volume of each balloon can be independently adjusted.

Pressure data are acquired while the first and second balloons are filled to different volumes, as prescribed by the particular application at hand, as indicated at step 306. For instance, first pressure data can be recorded using the pressure sensor transducers corresponding to the first balloon when the first balloon is in an expanded state, and second pressure data can be recorded using pressure sensor transducers corresponding to the second balloon when the second balloon is in an expanded state. As described above, the fill volume of the first and second balloons can be independently controlled, such that in some instances first and second pressure data may be recorded at the same time (e.g., when both the first and second balloons are in their respective expanded states), or first and/or second pressure data may be recorded independently (e.g., when only one of the first or second balloons is in its respective expanded state). As an example, the volume of the second balloon can be independently controlled in order to elicit a secondary peristalsis.

As one non-limiting example, steps 304 and 306 can implement the following filling and measurement protocol. The large (i.e., first) balloon can be filled to 70% max fill volume, after which pressure tracings can be monitored for one minute. The proximal small (i.e., second) balloon can then be rapidly filled to a max fill volume, or until a selected pressure threshold is measured. Pressure tracings are then monitored for one minute and the second balloon is then rapidly emptied. The first balloon can then be filled to 85% max fill volume and pressure tracings monitored for one minute. The second balloon can then again be rapidly filled to a max fill volume, or until a selected pressure threshold is measured. Pressure tracings are then monitored for one minute and the second balloon is then rapidly emptied. The first balloon can then be filled to 100% max fill volume and pressure tracings monitored for one minute. The second balloon can then once again be rapidly filled to a max fill volume, or until a selected pressure threshold is measured. Pressure tracings are then monitored for one minute and the second balloon is then rapidly emptied. The first balloon can then be emptied to 85% of its max fill volume and pressure tracings monitored for one minute. The first balloon can then be emptied to 70% of its max fill volume and pressure tracings monitored for one minute.

Referring still to FIG. 3, the measured pressure data are then classified using a computer system, as indicated at step 308. As one example, the measured pressure data can be input to a classifier implemented with the computer system, which may include a classification algorithm implemented with the computer system. As another example, a trained machine learning algorithm can be accessed with the computer system and the pressure data can be input to the trained machine learning algorithm, generating output as classified pressure data, which may include classified patterns of pressure variations. The trained machine learning algorithm may include a neural network or other suitable machine learning algorithm, such as a support vector machine (“SVM”) algorithm, a k-means cluster, and so on.

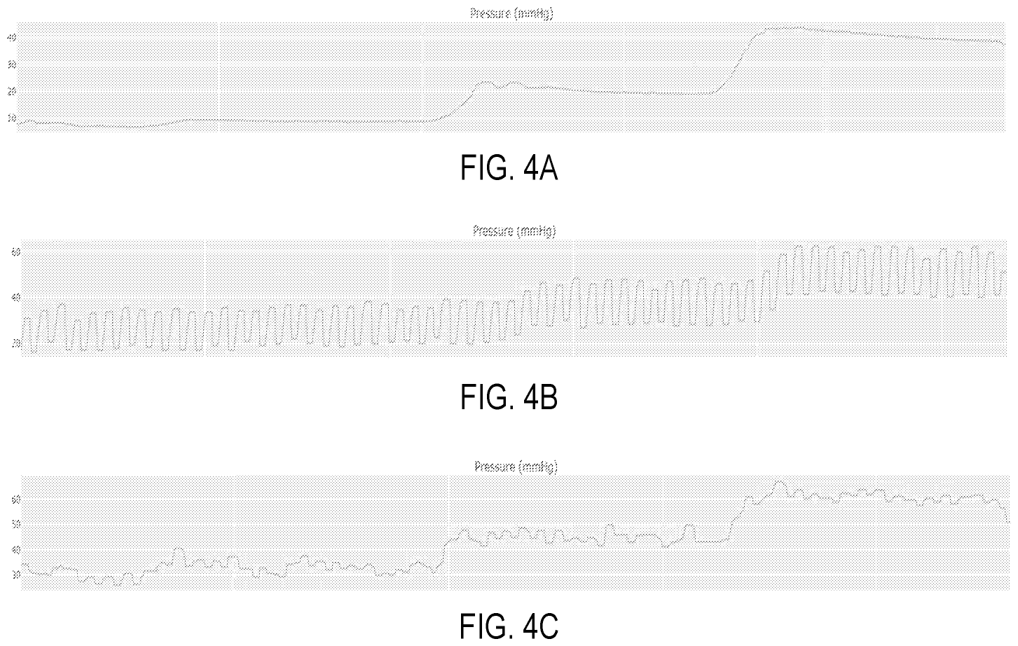

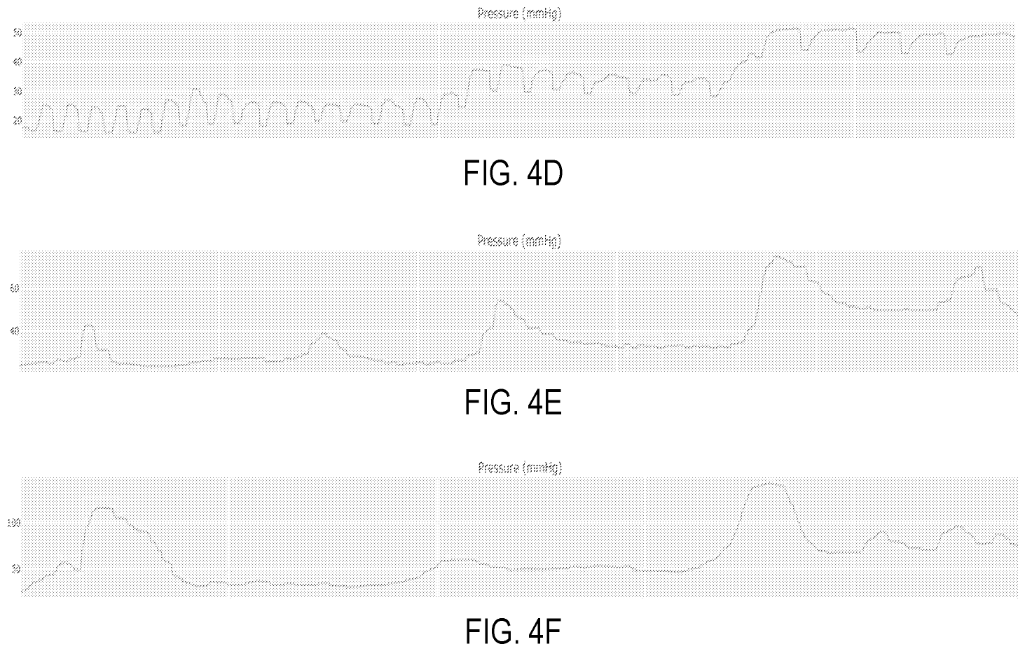

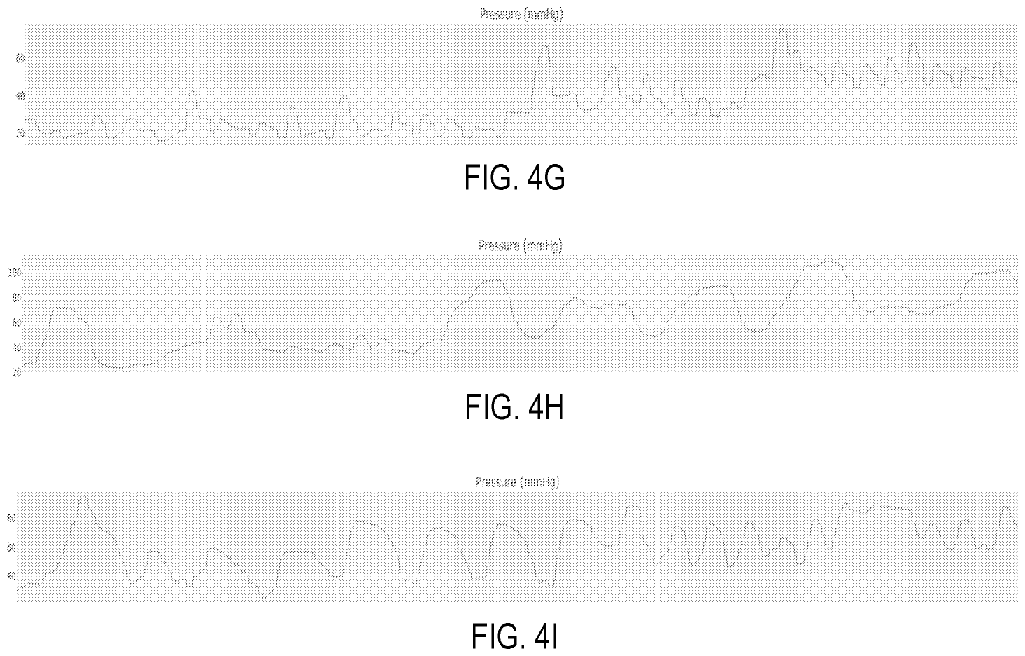

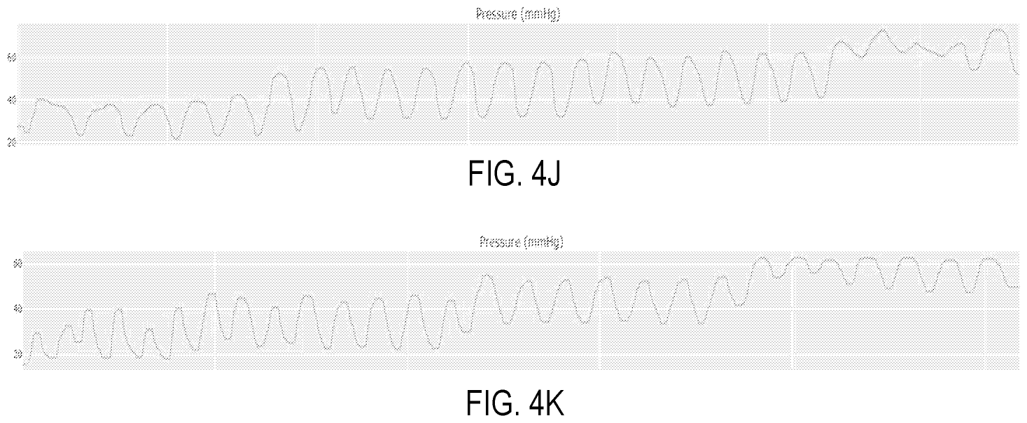

FIGS. 4A-4K illustrate examples of pressure profile patterns that can be used to construct or otherwise train a machine learning algorithm or model. FIG. 4A shows an example of a pressure pattern indicative of an absent contractile response (“ACR”) flat (i.e., dilated achalasia) pattern. FIG. 4B shows an example of a pressure pattern indicative of an ACR pattern with 1:1 respiratory rate (“RR”), which can be representative of scleroderma and/or treated achalasia. In this example, peak pressure synchronizes with esophagogastric junction (“EGJ”) opening. FIG. 4C shows an example of a pressure pattern indicative of an impaired-disordered contractile response (“IDCR”) pattern with 1:1 RR, which can be representative of treated achalasia. In this example, peak pressure synchronizes with EGJ opening. FIG. 4D shows an example of a pressure pattern indicative of an ACR/IDCR pattern with 1:2 RR, which can be representative of gastroesophageal reflux disease (“GERD”). In this example, a functional lumen imaging probe (“FLIP”) measured pressure in peak-opposite. FIG. 4E shows an example of a pressure pattern indicative of an ACR/IDCR pattern, which can be representative of spastic-reactive lower esophageal sphincter (“LES”) to fill EGJ with borderline normal response. FIG. 4F shows an example of a pressure pattern indicative of an ACR/IDCR pattern, which can be representative of spastic-reactive LES to fill EGJ with a tight EGJ. The higher pressure indicated in this pattern can indicate a likelihood of achalasia. FIG. 4G shows an example of a pressure pattern indicative of a borderline contractile response (“BCR”) pattern having erratic antegrade contractions. FIG. 4H shows an example of a pressure pattern indicative of a BCR pattern that is borderline normal. FIG. 4I shows another example of a pressure pattern indicative of a BCR pattern than is borderline normal. FIGS. 4J and 4K show examples of pressure patterns indicative of a normal pattern. As noted above, these pressure profiles and patterns can be used to construct and/or train a model for classification, including a machine learning algorithm or model, or other classifier algorithm or model.

Referring again to FIG. 3, from the classified pressure data, esophageal function data are then generated, as indicated at step 310. The esophageal function data indicate functional characteristics or properties of the subject's esophagus. As a non-limiting example, the esophageal function data may include an estimation of a pressure inversion point and measurements of the propagation of peristalsis in the esophagus. The pressure data, classified pressure data, and/or esophageal function data can then be stored for later use, or displayed to a user, as indicated at step 312.

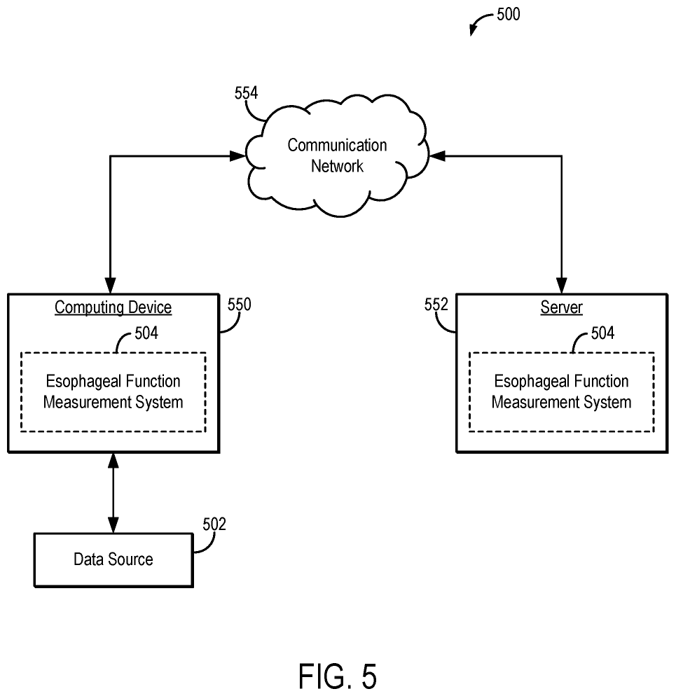

Referring now to FIG. 5, an example of a system 500 for measuring esophageal function in accordance with some embodiments of the systems and methods described in the present disclosure is shown. As shown in FIG. 5, a computing device 550 can receive one or more types of data (e.g., pressure data) from data source 502. In some embodiments, computing device 550 can execute at least a portion of an esophageal function measurement system 504 to generate esophageal function data from pressure data received from the data source 502.

Additionally or alternatively, in some embodiments, the computing device 550 can communicate information about data received from the data source 502 to a server 552 over a communication network 554, which can execute at least a portion of the esophageal function measurement system 504. In such embodiments, the server 552 can return information to the computing device 550 (and/or any other suitable computing device) indicative of an output of the esophageal function measurement system 504.

In some embodiments, computing device 550 and/or server 552 can be any suitable computing device or combination of devices, such as a desktop computer, a laptop computer, a smartphone, a tablet computer, a wearable computer, a server computer, a virtual machine being executed by a physical computing device, and so on. The computing device 550 and/or server 552 can also classify pressure data and generate esophageal function data therefrom.

In some embodiments, data source 502 can be any suitable source of pressure data, such as pressure sensor transducers on the baro-hydrometer device described in the present disclosure, another computing device (e.g., a server storing pressure data), and so on. In some embodiments, data source 502 can be local to computing device 550. For example, data source 502 can be incorporated with computing device 550 (e.g., computing device 550 can be configured as part of a device for capturing, scanning, and/or storing pressure data). As another example, data source 502 can be connected to computing device 550 by a cable, a direct wireless link, and so on. Additionally or alternatively, in some embodiments, data source 502 can be located locally and/or remotely from computing device 550, and can communicate data to computing device 550 (and/or server 552) via a communication network (e.g., communication network 554).

In some embodiments, communication network 554 can be any suitable communication network or combination of communication networks. For example, communication network 554 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 554 can be a local area network, a wide area network, a public network (e.g., the Internet), a private or semi-private network (e.g., a corporate or university intranet), any other suitable type of network, or any suitable combination of networks. Communications links shown in FIG. 5 can each be any suitable communications link or combination of communications links, such as wired links, fiber optic links, Wi-Fi links, Bluetooth links, cellular links, and so on.

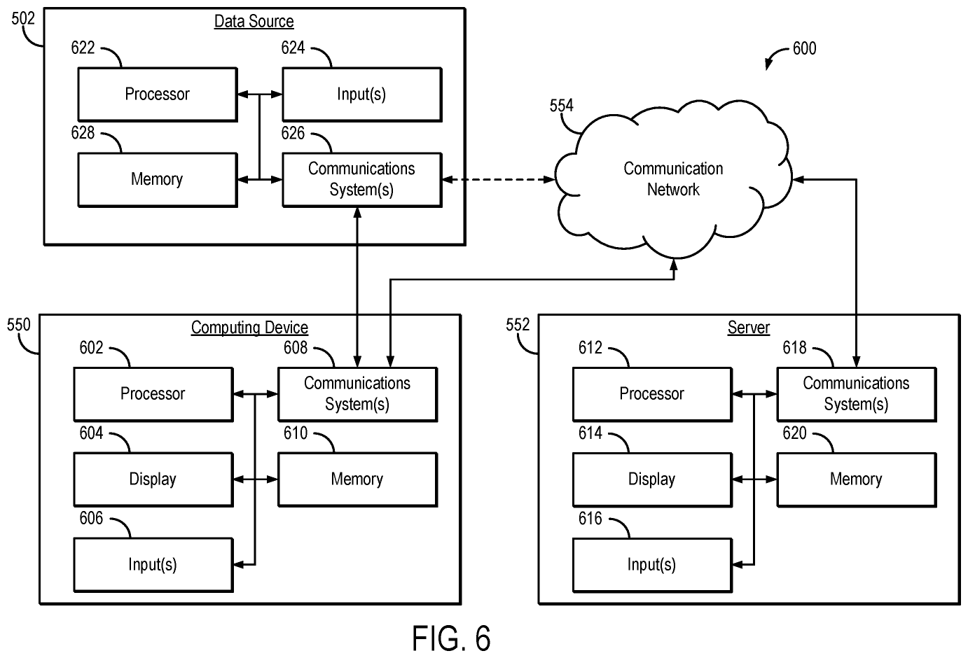

Referring now to FIG. 6, an example of hardware 600 that can be used to implement data source 502, computing device 550, and server 552 in accordance with some embodiments of the systems and methods described in the present disclosure is shown. As shown in FIG. 6, in some embodiments, computing device 550 can include a processor 602, a display 604, one or more inputs 606, one or more communication systems 608, and/or memory 610. In some embodiments, processor 602 can be any suitable hardware processor or combination of processors, such as a central processing unit (“CPU”), a graphics processing unit (“GPU”), and so on. In some embodiments, display 604 can include any suitable display devices, such as a computer monitor, a touchscreen, a television, and so on. In some embodiments, inputs 606 can include any suitable input devices and/or sensors that can be used to receive user input, such as a keyboard, a mouse, a touchscreen, a microphone, and so on.

In some embodiments, communications systems 608 can include any suitable hardware, firmware, and/or software for communicating information over communication network 554 and/or any other suitable communication networks. For example, communications systems 608 can include one or more transceivers, one or more communication chips and/or chip sets, and so on. In a more particular example, communications systems 608 can include hardware, firmware and/or software that can be used to establish a Wi-Fi connection, a Bluetooth connection, a cellular connection, an Ethernet connection, and so on.

In some embodiments, memory 610 can include any suitable storage device or devices that can be used to store instructions, values, data, or the like, that can be used, for example, by processor 602 to present content using display 604, to communicate with server 552 via communications system(s) 608, and so on. Memory 610 can include any suitable volatile memory, non-volatile memory, storage, or any suitable combination thereof. For example, memory 610 can include RAM, ROM, EEPROM, one or more flash drives, one or more hard disks, one or more solid state drives, one or more optical drives, and so on. In some embodiments, memory 610 can have encoded thereon, or otherwise stored therein, a computer program for controlling operation of computing device 550. In such embodiments, processor 602 can execute at least a portion of the computer program to present content (e.g., images, user interfaces, graphics, tables), receive content from server 552, transmit information to server 552, and so on.

In some embodiments, server 552 can include a processor 612, a display 614, one or more inputs 616, one or more communications systems 618, and/or memory 620. In some embodiments, processor 612 can be any suitable hardware processor or combination of processors, such as a CPU, a GPU, and so on. In some embodiments, display 614 can include any suitable display devices, such as a computer monitor, a touchscreen, a television, and so on. In some embodiments, inputs 616 can include any suitable input devices and/or sensors that can be used to receive user input, such as a keyboard, a mouse, a touchscreen, a microphone, and so on.

In some embodiments, communications systems 618 can include any suitable hardware, firmware, and/or software for communicating information over communication network 554 and/or any other suitable communication networks. For example, communications systems 618 can include one or more transceivers, one or more communication chips and/or chip sets, and so on. In a more particular example, communications systems 618 can include hardware, firmware and/or software that can be used to establish a Wi-Fi connection, a Bluetooth connection, a cellular connection, an Ethernet connection, and so on.

In some embodiments, memory 620 can include any suitable storage device or devices that can be used to store instructions, values, data, or the like, that can be used, for example, by processor 612 to present content using display 614, to communicate with one or more computing devices 550, and so on. Memory 620 can include any suitable volatile memory, non-volatile memory, storage, or any suitable combination thereof. For example, memory 620 can include RAM, ROM, EEPROM, one or more flash drives, one or more hard disks, one or more solid state drives, one or more optical drives, and so on. In some embodiments, memory 620 can have encoded thereon a server program for controlling operation of server 552. In such embodiments, processor 612 can execute at least a portion of the server program to transmit information and/or content (e.g., data, images, a user interface) to one or more computing devices 550, receive information and/or content from one or more computing devices 550, receive instructions from one or more devices (e.g., a personal computer, a laptop computer, a tablet computer, a smartphone), and so on.

In some embodiments, data source 502 can include a processor 622, one or more inputs 624, one or more communications systems 626, and/or memory 628. In some embodiments, processor 622 can be any suitable hardware processor or combination of processors, such as a CPU, a GPU, and so on. In some embodiments, the one or more inputs 624 are generally configured to pressure data, and can include an pressure sensor transducers on the baro-hydrometer device described in the present disclosure. Additionally or alternatively, in some embodiments, one or more inputs 624 can include any suitable hardware, firmware, and/or software for coupling to and/or controlling operations of a baro-hydrometer device. In some embodiments, one or more portions of the one or more inputs 624 can be removable and/or replaceable.

Note that, although not shown, data source 502 can include any suitable inputs and/or outputs. For example, data source 502 can include input devices and/or sensors that can be used to receive user input, such as a keyboard, a mouse, a touchscreen, a microphone, a trackpad, a trackball, and so on. As another example, data source 502 can include any suitable display devices, such as a computer monitor, a touchscreen, a television, etc., one or more speakers, and so on.

In some embodiments, communications systems 626 can include any suitable hardware, firmware, and/or software for communicating information to computing device 550 (and, in some embodiments, over communication network 554 and/or any other suitable communication networks). For example, communications systems 626 can include one or more transceivers, one or more communication chips and/or chip sets, and so on. In a more particular example, communications systems 626 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 628 can include any suitable storage device or devices that can be used to store instructions, values, data, or the like, that can be used, for example, by processor 622 to control the one or more inputs 624, and/or receive data from the one or more inputs 624; to generate esophageal function data from pressure data; present content (e.g., images, a user interface) using a display; communicate with one or more computing devices 550; and so on. Memory 628 can include any suitable volatile memory, non-volatile memory, storage, or any suitable combination thereof. For example, memory 628 can include RAM, ROM, EEPROM, one or more flash drives, one or more hard disks, one or more solid state drives, one or more optical drives, and so on. In some embodiments, memory 628 can have encoded thereon, or otherwise stored therein, a program for controlling operation of data source 502. In such embodiments, processor 622 can execute at least a portion of the program to generate esophageal function data, transmit information and/or content (e.g., data, images) to one or more computing devices 550, receive information and/or content from one or more computing devices 550, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A baro-hydrometer device, comprising:
a catheter extending from a first end to a second end along a longitudinal axis;
a first balloon arranged at the first end of the catheter;
a second balloon arranged at the second end of the catheter, wherein the second balloon is smaller than the first balloon;
a first infusion port fluidically coupling the first balloon to the catheter;
a second infusion port fluidically coupling the second balloon to the catheter; and
a plurality of pressure sensors arranged on the first balloon and the second balloon.

2. The baro-hydrometer device of claim 1, wherein the first balloon and the second balloon have a common maximum diameter.

3. The baro-hydrometer device of claim 1, wherein the second balloon has a shorter length along the longitudinal axis than the first balloon.

4. The baro-hydrometer device of claim 1, wherein the catheter comprises a first channel providing fluid connection to the first infusion port, and a second channel providing fluid connection to the second infusion port.

5. The baro-hydrometer device of claim 4, wherein the first channel is not in fluid communication with the second channel such that a fill volume of the first balloon can be adjusted independent from a fill volume of the second balloon.

6. The baro-hydrometer device of claim 1, wherein the plurality of pressure transducers comprises at least two pressure transducers coupled to the first balloon and at least one pressure transducer coupled to the second balloon.

7. The baro-hydrometer device of claim 1, wherein the first infusion port comprises a plurality of first infusion ports that fluidically couple the first balloon to the catheter.

8. The baro-hydrometer device of claim 1, wherein the first balloon is composed of polyurethane.

9. A method for measuring esophageal function in a subject, the method comprising:
(a) positioning a catheter within an esophagus of a subject, wherein the catheter includes a first balloon having a pressure sensor coupled thereto and a second balloon having a pressure sensor coupled thereto;
(b) measuring pressure data with the pressure sensors coupled to the first balloon and the second balloon while independently controlling a fill volume of the first balloon and a fill volume of the second balloon;
(c) generating classified pressure data by inputting the pressure data to a classifier implemented with a computer system; and
(d) generating esophageal function data from the classified pressure data using the computer system.

10. The method of claim 9, wherein independently controlling the fill volume of the first balloon and the fill volume of the second balloon comprises adjusting the fill volume of the second balloon to elicit a secondary peristalsis in the esophagus of the subject.

11. The method of claim 9, wherein the fill volume of the first balloon and the fill volume of the second balloon are independently adjusted by adjusting a first volume of fluid in the first balloon and a second volume of fluid in the second balloon.

12. The method of claim 11, wherein the fluid is air.

13. The method of claim 11, wherein the fluid is saline.

14. The method of claim 11, wherein the first balloon is filled with a first fluid and the second balloon is filled with a second fluid that is different from the first fluid.

15. The method of claim 14, wherein the second fluid is air.

16. The method of claim 14, wherein the first fluid is saline.

17. The method of claim 9, wherein generating the classified pressure data comprises accessing a trained machine learning algorithm with the computer system and inputting the pressure data to the trained machine learning algorithm, generating output as the classified pressure data.

18. The method of claim 17, wherein the trained machine learning algorithm is trained on training data comprising pressure profiles indicative of different contractile response patterns.
